(19) United States
(12) Patent Application Publication
(10) Pub. No.: US 2002/0156420 A1
(43) Pub. Date: Oct. 24, 2002

(54) Fluid delivery cannula technology

(76) Inventors: Mark L. Anderson, Spring Valley, WI (US); Sylvan L. Johnson, Woodville, WI (US)

Correspondence Address: SKINNER & ASSOCIATES, 619 SECOND STREET, SUITE 201, HUDSON, WI, 54016, US

(21) Appl. No.: 09/839,579

(22) Filed: Apr. 20, 2001

(51) Int. Cl. H05B 1/00; A61F 7/12

(52) U.S. Cl. 604/113

(57) ABSTRACT

A medical fluid dispersing cannula for use in spraying and irrigation applications. The cannula comprises a cannula member, a plurality of ports, and an adapter. The cannula member has a lumen, a proximal end, and a curved, integrally formed distal end. The cannula member has a normal central longitudinal axis. The cannula member has an elongated, thin tubular configuration. The cannula member is flexible and constructed of a polymeric material. Preferably three ports in the cannula member are disposed on the curved distal end thereof, the ports being circular and disposed on the curved distal end so that they diverge with respect to each other and with respect to the central longitudinal axis. The luer adapter is connected to the proximal end of the cannula member for connection to a fluid source and to provide communication with the lumen of the cannula member and to the ports. Fluid entering the adapter is communicated through the lumen and is widely dispersed via the divergent ports.

### CROSS-REFERENCE TO RELATED APPLICATIONS, IF ANY

Not applicable.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### BACKGROUND OF THE INVENTION

1. Field of the Invention

The present invention relates, generally, to medical devices. More particularly, the invention relates to a medical fluid delivery cannula. Most particularly, the invention relates to a medical cannula with spray or irrigation capability that is especially useful for nasal flushing, wound irrigation or therapy, intranasal spray vaccination, or topical medicament spray delivery applications.

2. Background Information

The background technology, in general, includes apparatus and methods for dispensing fluids, including liquid medicaments. Some devices have spray or atomizing tips. Devices having tips are used to deliver fluid medicaments topically and intranasally.

This technology is believed to have significant limitations and shortcomings, including being cumbersome to use. For at least this reason, a need exists for the present invention.

This invention provides a spray cannula which is believed to fulfil the need and to constitute an improvement over the background technology by providing a spray or irrigation cannula which can be used for a variety of applications including, but not limited to flushing, irrigation, medicament or vaccine delivery, topically or intranasally.

### BRIEF SUMMARY OF THE INVENTION

The present invention provides a cannula which is useable to spray or irrigate. It can be used for a variety of applications including, but not limited to flushing, irrigation, vaccination, or medicament delivery, either topically or intranasally. The cannula projects fluids such as liquid medicament to multiple sites in a desired environment, such as a nasal cavity or a wound, via divergent ports.

In a basic embodiment, the invention provides a cannula comprising a cannula member having a lumen and a curved distal end; at least two ports in the cannula member disposed on the curved distal end thereof; and an adapter connected on the cannula member, for connection to a fluid source and to provide communication with the lumen of the cannula member.

In a further embodiment, the invention provides a medical fluid dispersing cannula for use in spraying and irrigation applications, comprising:

a. a cannula member having a lumen, a proximal end, and a curved, integrally formed distal end, the cannula member having a normal central longitudinal axis, the cannula member having a unitary body including the proximal end and the distal end, the body having an elongated, thin tubular configuration, the cannula member being flexible and constructed of a polymeric material;

b. at least three ports in the cannula member disposed on the curved distal end thereof, the ports being circular and disposed on the curved distal end so that they diverge with respect to each other and with respect to the central longitudinal axis; and

c. a luer adapter connected to the proximal end of the cannula member, for connection to a fluid source and to provide communication with the lumen of the cannula member and to the ports, whereby fluids entering the adapter is communicated through the lumen and is widely dispersed via the divergent ports.

The features, benefits and objects of this invention will become clear to those skilled in the art by reference to the following description, claims and drawings.

### BRIEF DESCRIPTION OF THE SEVERAL VIEWS OF THE DRAWING

FIG. 1 is a perspective view, partially exploded, of the medical fluid delivery cannula of the present invention.

FIG. 2 is a front view of a cannula member portion of the medical fluid delivery cannula of the present invention, showing some interior structure of the portion.

FIG. 3 is a distal end view of the cannula member portion shown in FIG. 2.

FIG. 4 is a detailed view of a portion of the cannula member portion shown in FIG. 2.

FIG. 5 is a detailed view of the cannula member portion shown in FIG. 3.

FIG. 6 is a distal end view of an adapter portion of the medical fluid delivery cannula.

FIG. 7 is a side view of the adapter portion shown in FIG. 6.

FIG. 8 is a proximal end view of the adapter portion shown in FIGS. 6 and 7.

FIG. 9 is an end view of a lock cap portion of the medical fluid delivery cannula.

FIG. 10 is a side view of the lock cap portion shown in FIG. 9.

FIG. 11 is an opposite end view of the lock cap portion shown in FIGS. 9 and 10.

### DETAILED DESCRIPTION

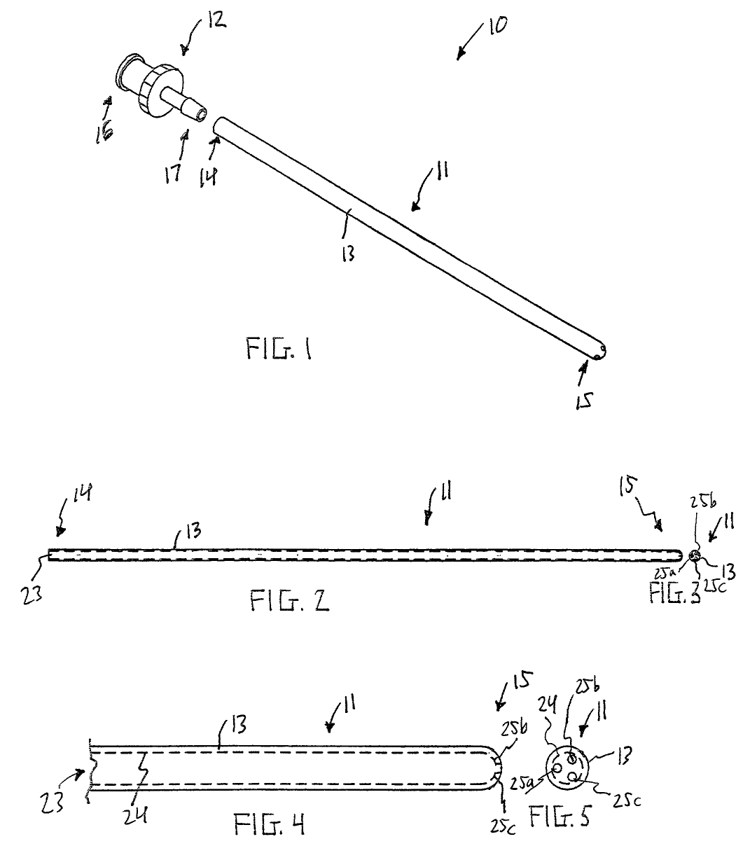

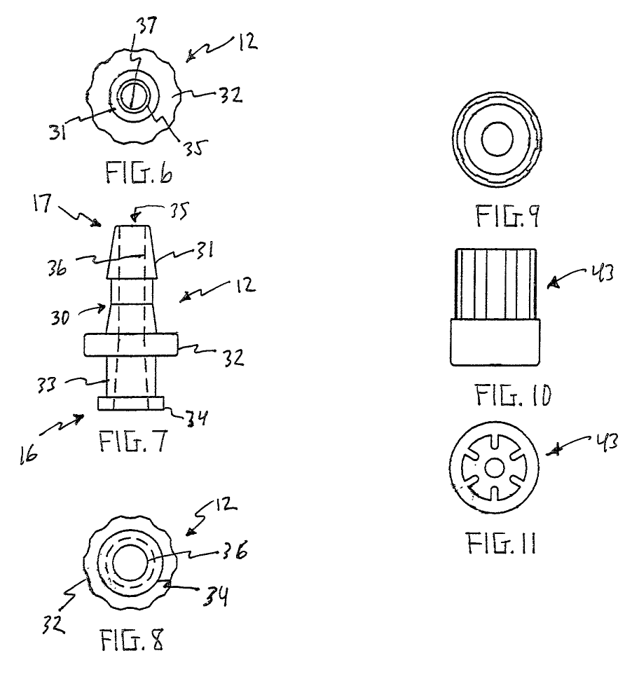

FIGS. 1-11 show an example of the preferred embodiment of the medical fluid delivery cannula of the present invention is illustrated and generally indicated by the reference numeral 10.

Referring to FIG. 1, the cannula apparatus 10 generally comprises a cannula member 11 and an adapter member 12. The cannula member 11 has a proximal end 14 and a distal end 15. The adapter member 12 has a proximal end 16 and a distal end 17. The distal end 17 of the adapter member 12 sealingly mates with the proximal end 14 of the cannula member 11. The free proximal end 16 of the adapter member 12 is adapted to interconnect with a fluid source (not shown), for example one which provides liquid medicaments for human or veterinary medical purposes. The distal end 15 of the cannula member 11 is adapted to be disposed to deliver fluid to the environment, for example a human patient or an animal, via cooperation which will be described in detail below with the other elements of the cannula member 11 and the adapter member 12. The cannula 10 is particularly well suited for intranasal delivery of medicaments.

Referring also to FIGS. 2-5, the cannula member 11 generally has an elongated, relatively thin, cylindrical structure. The cannula member 11 has tubular body 13 which is hollow, there by providing an interior lumen 23 which is defined by the interior wall 24 of the body 13. The lumen 23 is open at the proximal end 14 of the cannula member 11 such that it matingly receives the distal end 17 structure of the adapter member 12. The distal end 15 of the cannula member 11 has a rounded configuration. In the embodiment shown, three ports 25a, b and c are disposed at predetermined positions on the distal end 15. At least two ports are necessary to obtain desirable fluid dispersal. The ports 25 extend through the body 13 such that they are communicatively connected with the lumen 23. As is best shown in FIG. 5, preferably, the ports 25 have a circular configuration and are aligned so that they are evenly spaced with respect to each other and so that the outwardly facing portions of their respective peripheries are disposed proximate the inner wall 24 of the lumen 23. As is best shown in FIG. 4, as result of the curved distal end 15 of the body 13, and the orientation of the ports 25 thereon, at least a portion of each port 25a-c diverges from the other ports, and with respect to a central longitudinal axis of the device 10 which is defined when the cannula member 11 is in a normal, unflexed or straight state.

The cannula member 11 body 13 is constructed of a polymeric material. A preferred material is PVC. Alternatively, silicon material may be used. The material is flexible having a preferred durometer of 90 Shore A. In one embodiment for use in delivering medicaments intranasally, the cannula member 11 has a length of approximately six inches (15.2 cm) and an outside diameter of approximately ⅛ inches (0.3 cm). The lumen 23 has an inside diameter of approximately 1/16 inches. The ports 25 have a diameter of approximately 0.02 inches. The cannula member 11 has a unitary structure with the ported distal end 15 of the cannula member 11 is formed unitarily with the entire body 13.

Referring to FIGS. 6-8, the adapter member 12 generally has a luer lock type structure. The adapter member 12 comprises a distal stem 30 which has a tapered end 31, a circular hub 32, and a proximal stem 33 with a threaded end 34. The adapter member 12 is also hollow and provides a lumen 35 defined by an inside wall 36. The distal stem 30 with a tapered end 31 sealingly mates and forms a friction fit with the open end of the inner lumen 23 at the proximal end of the cannula member 11. The hub 32 preferably has a knurled circumferential surface and is adapted to be grasped and manipulated by the user's hand for connection to a fluid source (not shown). The end 34 has a predetermined diameter and threads for secure, fluid tight connection to the fluid source. The proximal and distal stems 30 and 33 may have approximately equivalent respective inside and outside dimensions as is shown or, alternatively, the dimensions may be varied depending upon the particular required dimensional adaptation required given the dimensions of the fluid source and cannula member.

The adapter member 12 is constructed of a rigid polymeric material. A preferred material is polyethylene.

Referring to FIGS. 9-11, a lock cap 43 is shown for mating with the adapter member 12.

In use, the adapter member 12 is connected by a user to a fluid source which has a complementary mating means. The fluid source provides a fluid such as a liquid medicament. The fluid source may further have means to control the flow of fluid to the cannula 10. The distal end 15 of the cannula member 11 is directed by the user to a target area at which fluid is desired to be provided by the user. The flexible cannula member 12 permits accurate, safe and comfortable direction to the target area. Fluid passes through the adapter member 12, into the lumen 23 of the cannula member 12, and out of the ports 25 at the distal end of the cannula member 12. Due to the configuration of the ports 25, and their placement on the curved distal end 15 discussed above, the ports 25 disperse the fluid at the target area.

The descriptions above and the accompanying drawings should be interpreted in the illustrative and not the limited sense. While the invention has been disclosed in connection with the preferred embodiment or embodiments thereof, it should be understood that there may be other embodiments which fall within the scope of the invention as defined by the following claims. Where a claim, if any, is expressed as a means or step for performing a specified function it is intended that such claim be construed to cover the corresponding structure, material, or acts described in the specification and equivalents thereof, including both structural equivalents and equivalent structures, material-based equivalents and equivalent materials, and act-based equivalents and equivalent acts.

What is claimed is:

1. A cannula, comprising:
a cannula member having a lumen and a curved distal end;
at least two ports in the cannula member disposed on the curved distal end thereof; and
an adapter connected on the cannula member, for connection to a fluid source and to provide communication with the lumen of the cannula member.

2. The cannula of claim 1, wherein the cannula member has a elongated, thin, tubular configuration.

3. The cannula of claim 1, wherein the cannula member is constructed of a polymeric material.

4. The cannula of claim 3, wherein the polymeric material is selected from the group of materials consisting of PVC and silicone.

5. The cannula of claim 1, wherein the cannula member is flexible.

6. The cannula of claim 5, wherein and exhibits a durometer reading of approximately 90 Shore A.

7. The cannula of claim 1, wherein the distal end of the cannula member is integrally formed with cannula member.

8. The cannula of claim 1, wherein the cannula has three ports.

9. The cannula of claim 8, wherein each port has a curvilinear configuration.

10. The cannula of claim 9, wherein the curvilinear configuration is a semi-circle.

11. The cannula of claim 8 wherein the cannula member has a normal central longitudinal axis, and wherein the ports are disposed on the curved distal end so that they diverge with respect to each other and with respect to the central longitudinal axis.

12. The cannula of claim 1, wherein the cannula member has a proximal end, and wherein said adapter is connected to said proximal end.

13. The cannula of claim 12, wherein said adapter is friction fit connected to the proximal end.

14. The cannula of claim 1, wherein the adapter is a luer lock type adapter.

15. The cannula of claim 1, further comprising a cap for connection to the adapter.

16. The cannula of claim 15, wherein the cap is a luer lock type cap.

17. The cannula of claim 1, for use in dispersing fluids for medical applications.

18. A fluid dispersing cannula, comprising:
a. a cannula member having a lumen and a curved, integrally formed distal end, the cannula member having a normal central longitudinal axis;
b. at least three ports in the cannula member disposed on the curved distal end thereof, the ports being disposed on the curved distal end so that they diverge with respect to each other and with respect to the central longitudinal axis; and
c. an adapter connected on the cannula member, for connection to a fluid source and to provide communication with the lumen of the cannula member.

19. A medical fluid dispersing cannula for use in spraying and irrigation applications, comprising:
a. a cannula member having a lumen, a proximal end, and a curved, integrally formed distal end, the cannula member having a normal central longitudinal axis, the cannula member having a unitary body including the proximal end and the distal end, the body having an elongated, thin tubular configuration, the cannula member being flexible and constructed of a polymeric material;
b. at least three ports in the cannula member disposed on the curved distal end thereof, the ports being circular and disposed on the curved distal end so that they diverge with respect to each other and with respect to the central longitudinal axis; and
c. a luer adapter connected to the proximal end of the cannula member, for connection to a fluid source and to provide communication with the lumen of the cannula member and to the ports, whereby fluids entering the adapter is communicated through the lumen and is widely dispersed via the divergent ports.
